(19) United States
(12) Patent Application Publication
(10) Pub. No.: US 2003/0199085 A1
(43) Pub. Date: Oct. 23, 2003

(54) Transportation of stem cells

(76) Inventors: Abraham Berger, Givataim (IL); Ben Zion Haim, Holon (IL); Baruch Meirovich, Hod Hasharon (IL); Avri Hazan, Givataim (IL)

Correspondence Address: NATH & ASSOCIATES, 1030 15th STREET, 6TH FLOOR, WASHINGTON, DC, 20005, US

(21) Appl. No.: 10/126,947

(22) Filed: Apr. 22, 2002

(51) Int. Cl. A61K 48/00; A61M 5/00; C12N 5/08

(52) U.S. Cl. 435/366; 424/93.2; 604/246

(57) ABSTRACT

A method for delivery, a catheter extension, connection and arresting valves and monitoring and pressure discharge systems for use in the field of cells transplantation or regeneration therapy, or implantation

### FIELD OF THE INVENTION

The invention relates to cells delivery for use for example in cells transplantation therapy.

### BACKGROUND OF THE INVENTION

Urban embryonic stem (ES) cells are pluripotent cell lines that have been derived from the inner cell mass (ICM) of blastocytes stage embryos or from hematopoietic stem cells. They are characterized by their ability to be propagated indefinitely in culture as undifferentiated cells with a normal karyotype and can be induced to differentiate in vitro into various cell types. Thus, human ES cells promise to serve as an, unlimited cell source for transplantation in numerous pathologies.

The fundamental strategy to exploit this discovery is to grow differentiated cells in a laboratory dish that are suitable for implantation into a patient by starting e with undifferentiated cells. The idea is either to treat the cells in culture medium to nudge them toward the desired differentiated cell type before implantation, or to implant them directly and rely on signals inside the body to direct their maturation into the desired kind of cells.

The present invention is directed to provide a method of delivering cells, such as ES cells to a targeted tissue for transplantation or between different cites.

### SUMMARY OF THE INVENTION

The present invention is particularly intended for a use in the field of cells transplantation therapy or regeneration therapy or implantation. In the context of the present invention, the term cells relates to stern cells from, any living organism, sources such as: pluripotent stern cells and more specifically to human embryonic pluripotent stem cells (e.g. differentiated cells to a specific tissue cloned cells), embryoid cells, cloned cells, a single cell or clumps or patch of cells, or any combination thereof. The term cells may also relate to fractions of cells e.g. DNA or RNA or cells which comprise additional material/markers such as magnetic marking. Cells in the present invention may also relate to embryo in an in vitro fertilization embryo transfer (IVF-ET) procedure.

In accordance with one aspect of the present invention, a method for depositing a flattened droplet on a surface described in WO 99/18872 may be used for the delivery of cell(s) containing culture medium to a desired site.

A “flattened droplet” in the context of the present invention can be demonstrated of standard 80 gram/m² A4 paper for use with ink jet printers, such paper constituting a partially absorbent surface on which a flattened droplet of the present invention has a projected surface area about three to six times larger than that of a naturally forming dome-like droplet. A “partially absorbentyurface” in the context of the present invention is one which absorbs a relatively insignificant volume of a naturally forming dome-like droplet over about 60 seconds. The flattening of a droplet as achieved by the method of the present invention is not by the relatively slow process of its being absorbed assuming it does not dry but rather as a consequence of its being effectively inflated by one or more bubbles of displacement gas controllably blown thereinto towards the end of its discharge which typically occurs over 5-20 seconds from an initial outward displacement of the microvolume of liquid (ML). The surface may be flat, inclined or even inverted and still maintain the droplet in its flattened shape by virtue of the prevailing surface tension therewith. In addition, the surface on which the droplet is placed may be cracked or even broken.

A “microvolume of liquid” (NL) in the context of the present invention is a volume of liquid in the microliter range, e.g., within the range of 0.01-5 μl, preferably within the range of 0.1-3.0 μl and particularly within the range of 0.3-2.0 μl. In the case of cells delivery procedure on a human subject, when the catheter is upwardly inclined, even though the discharge of culture medium is relatively slow, its volume should be so small as to avoid a downward trickle along the catbeter's outer surface.

In accordance with another aspect of the present invention, there is provided a catheter extension for use with the method of the first aspect of the present invention as well as wish any other cells delivering method. The catheter extension is adapted for a stationary installation, mainly inside the human body, or at any other site where a necessity exists to perform successive delivery of a plurality of cells to a targeted tissue. The catheter extension is installed at a desired orientation, preferably, with a possibility to adjust this orientation without withdrawing or essentially moving the tube, to repeatedly perform the implantation procedure in order to dispose the transplanted cells adjacent to one another. This movement is most preferably controlled via a control mechanism comprising for example) a carriage and a leading screw. However, the control may also be performed manually. The catheter extension is in the form of a tube adapted for sealed end-to-end connection with a catheter by means of any appropriate connector or valve, to avoid the need of insertion the catheter inside the tube and of controlling the position of the catheter relative to die proximal end of the tube.

In accordance with farther aspect of the present invention, there is provided a connection valve which provides connection between the extension tube and a catheter's outlet section, as well as between the catheter's outlet section and a catheter's inlet section, and is adapted to create a ML path from a distal end of the catheter's inlet section where the ML is aspirated into the catheter, to the catheter's outlet section and farther inside the outlet section where the ML is arrested at a predetermined location spaced from the proximal end of the catheter, and is then displaced outwardly from the catheter's outlet section and moved, via said connecting valve, into the extension tube to be finally discharged from its distal end.

In accordance with still another aspect of the present invention, there is provided an arresting valve adapted to control tie inward movement of a ML inside the catheter and, particularly, to provide the full arrest of the E movement before it reaches a predetermined arresting location spaced from the proximal end of the catheter. After such arrest, the outward movement of ML for its withdrawal from the catheter starts, for the discharge of two ML at a targeted disposition. The arresting valve is connected to the catheter at a location between the proximal end of the catheter and the arresting location of the ML, and it is designed to be opened to the atmosphere, to thereby cancel the pressure differential between the proximal and distal ends of die catheter, chic contains the ML, thereby filly stopping the inside movement of the ML.

In accordance with still another aspect of the present invention, there is provided a monitoring system for the delivery of cells to a Closed Volume (CV). A Closed Volume is for example a womb, an epidural space, parts of the brain etc, The monitoring device informs of any problems such as folding or soft tissue blockade inside the catheter.

In accordance with a further aspect of the present invention, there is provided a system which includes a catheter and a guide enabling the discharge of pressure from the CV. The guide which is partially inserted to the body, allows the catheter to slide directly and accurately to the target tissue. The guide can be used for example in an Embryo Transfer (E-T) procedure, in which the guide is inserted to the cervix for reducing the pressure created at the distal end of the NML.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 is a pictorial view of apparatus for depositing a flattened droplet comprising a cell(s) on a surface in accordance with one aspect of the present inventions;

FIGS. 2A-2L illustrates operation of the apparatus of FIG. 1 for depositing the flattened droplet comprising a cell(s) on the surface;

FIG. 3 is a pictorial view of a pump in accordance with the present invention, and

FIGS. 4 and 5 are cross sectional views of the pump of FIG. 3 along lines A-A and 13-B in FIG. 3, respectively.

FIGS. 6A to 6C schematically illustrate a catheter extension in accordance *with another aspect of the present invention, and the manner of its operation.

FIG. 7 illustrates a surface covered with a number of droplets.

FIGS. 8A to 8C illustrate a system with connection and arresting valves in accordance with still other aspects of the present invention.

FIG. 9A illustrates a delivery system with a monitoring device, according to a further aspect of the present invention.

FIG. 9B illustrates physical forces influencing a NL during the delivery process.

FIG. 10 graphically illustrates the variations of pressure during the delivery of a ML.

FIG. 11 is a cross section of the catheter inside the guide along the line A-A in FIG. 9A.

### DETAILED DESCRIPTION OF THE INVENTION

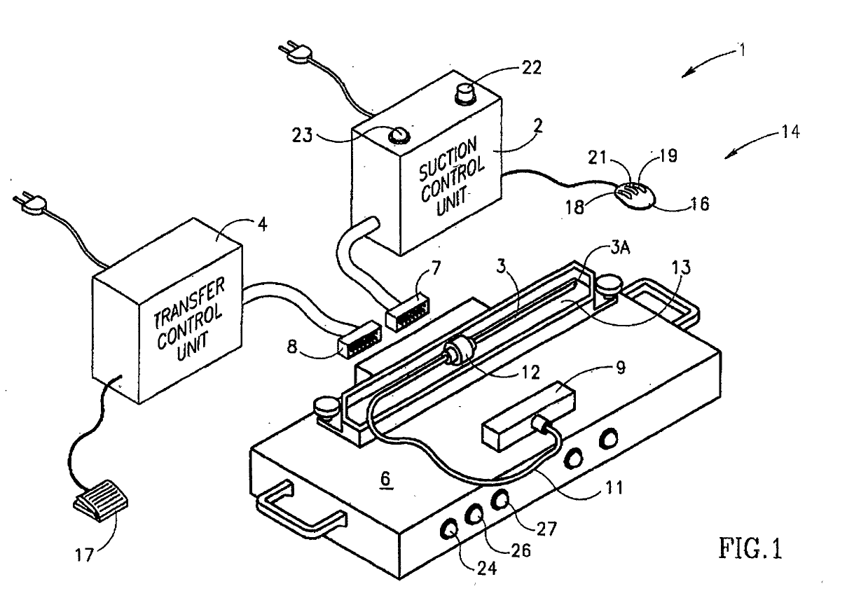

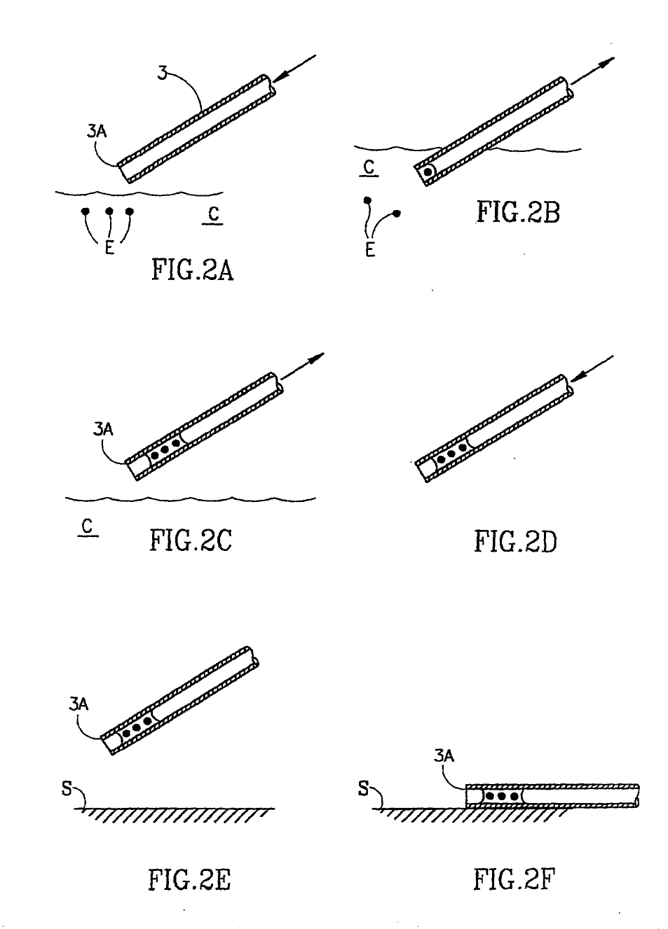

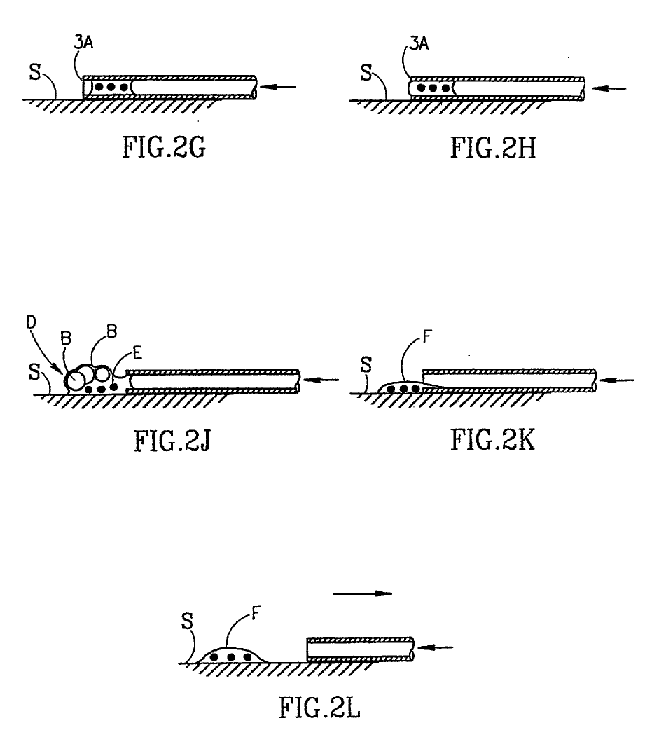

With reference now to FIGS. 1 and 2, apparatus 1 is employed for depositing a flattened droplet F on a partially absorbent surface S, for example, on a patient's liver or heart muscle. Apparatus 1 includes a suction control, unit 2 normally permanently located in a laboratory for the preparation of cells, carrying catheter 3 in the form of a transfer tube, a transfer control unit 4 normally permanently located in a treatment room where the therapy procedure is carried out and a portable casing 6 for consecutive connection to the suction control unit 2 and the transfer control unit 4 by means of connectors 7 and 8. The casing 6 includes a pneumatic system 9 which is permanently connected to the catheter 3 during an entire cells delivery procedure via suitable air tubing 11 and an air filter 12. The casing 6 also has a receptacle 13 for accommodating tube catheter 3 during its transport from the laboratory to the treatment room.

The pneumatic system 9 is under a control mechanism. 14 including a computer mouse 16 for controlling the suction control unit 2 for initiating a user controlled suction mode to prepare the catheter 3 with a microvolume of cell(s) containing culture medium and a foot pedal 17 for controlling the transfer control unit 4 for initiating a user initiated automated delivery mode for depositing the flattened droplet F on the surface S. The computer mouse 16 has an upstroke control 18 for drawing an incoming flow of displacement gas into the pneumatic system 9 from the catheter 3, a downstroke control 19 for issuing an outgoing flow of displacement gas from the pneumatic system 9 into the catheter 3 and optionally a speed control 21 for controlling the flow rate of the displacement gas either from or into the pneumatic system 9. The suction control unit 2 is also provided with a reset button 22 for priming the pneumatic system 9 for a pre-suction mode of issuing an outgoing flow of displacement gas as indicated by a READY indicator light 23 prior to the preparation of the catheter 3. The different stages of the automatic delivery mode are indicated by a READY indicator light 24, a GO indicator light 26 and a DONE indicator light 27.

In operation, the casing 6 is initially connected to the suction control unit 2 and the catheter 3 is connected to the pneumatic system 9 via the air tubing 11 and the filter 12. An operator presses the reset button 22 whereupon a lit READY indicator light 23 indicates an outgoing flow of displacement gas creating a positive pressure within catheter 3 to prevent capillary forces drawing medium thereinto upon insertion of its distal end 3A into a vessel of culture medium C containing cell(s) E shown exaggerated in all FIGS. 2A-2L (see FIG. 2A). The operator inserts the distal end 3A into the vessel of culture medium C for aspirating about 0.3 to 0.6 μl micro-volume of culture medium containing the cell(s) E into the catheter 3 (see FIG. 2B). Once the cell(s) is clearly seen to be close to the catheter's distal] end 3A, the rate of aspiration of culture medium may be increased by depressing the speed control 21. If a single cell is to be transferred, distal end 3A is then be removed from the culture medium otherwise additional cells may be captured as shown.

Once the catheter 3 contains one or more ceils, the operator withdraws its distal end 3A from the culture medium and then proceeds to depress the downstroke control 19 to slowly displace the microvolume of culture medium inwardly (see FIG. 2C) After the microvolume of culture medium has been inwardly displaced by about 5-15 mm from the catheter's distal end 3A, its motion is arrested by a momentary flow of displacement gas (see FIG. 2D) so that it finally comes to rest at a distance of about 10 mm (see FIG. 2F), thereby ensuring that it cannot be inadvertently lost during transportation of the casing 6 between the laboratory and the treatment room. An alternative arresting procedure may be employed as will be described in detail below. The catheter 3 is then placed in the receptacle 13 (see FIG. 1) during the transportation of the casing 6 from the laboratory to the treatment room.

For transfer of the cell(s), E onto the surface S, the catheter 3 is laid on the surface S (see FIG. 2F) whereupon a first depression on the foot pedal 17 causes the READY indicator light 24 to be lit indicating that the automatic delivery mode can be initiated. Thereafter, a second depression on the foot pedal 17 causes the GO indicator light 26 to be lit indicating that an outgoing flow of displacement gas is displacing the microvolume of culture medium towards the catheters distal end 3A (see FIG. 2G). The outgoing flow of displacement gas causes a concave shaped meniscus to be slowly formed which increases in size until it suddenly ruptures whereby most of the microvolume of culture medium is discharged as a droplet D on the surface S (see FIGS. 2H and 2J). The discharge is accompanied by one or more air bubbles B for effectively inflating the droplet D thereby considerably widening its projected surface area on the surface S to form the flattened droplet F whose shape is maintained by its prevailing surface tension with the surface S (see FIG. 2K).

The GO indicator light 26 is then extinguished indicating that the operator should slightly withdraw tie catheter 3 so as to detach it from the droplet F whilst at the same time there is an outgoing flow of displacement gas (see FIG. 2L,) in the case of the cell(s) delivery procedure, withdrawal is limited to between about 10-15 mm such that the catlheter's distal end 3A still lies along a subject's tissue. Finally, a further outgoing flow of additional displacement gas is provided so as to remove any culture medium which may remain in the catheter 3. The DONE indicator light 27 is then lit to indicate that the catheter 3 can be completely removed.

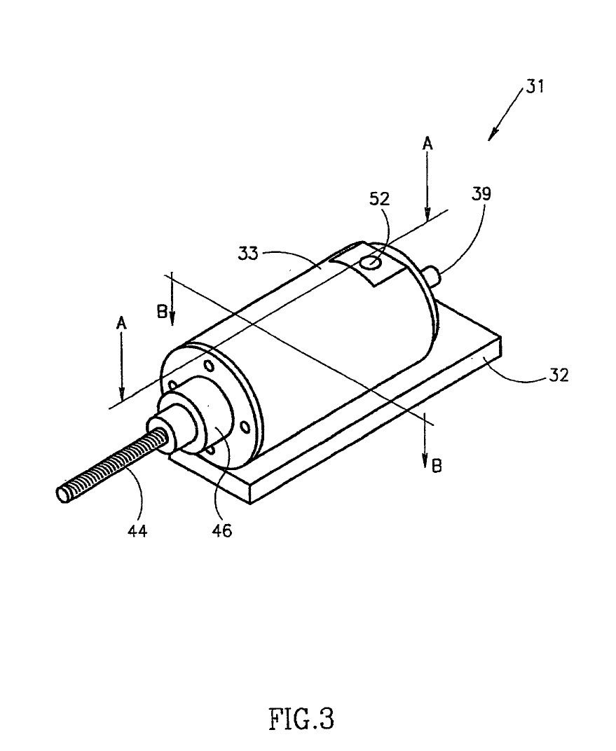

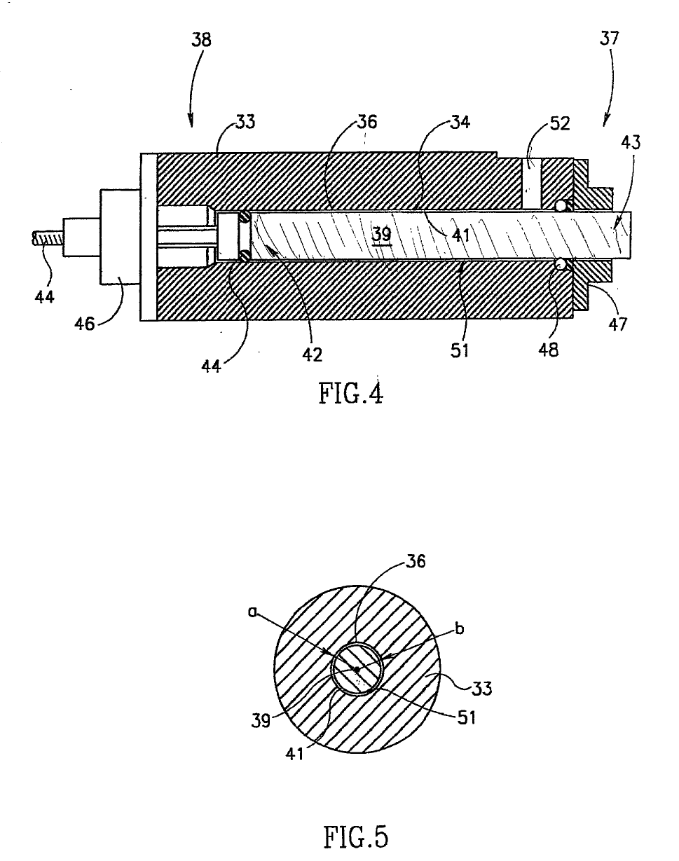

With reference now to FIGS. 3-5, a pump 31 constituting a pneumatic system for use with the apparatus 1 includes a base 32 with a housing 33 having a longitudinal right cylindrical through bore 34 with an internal peripheral surface 36 of a radius a and having first and second opposite ends 37 and 38. A right cylindrical slide rod 39 with an external peripheral surface 41 of a radius b and first and second opposite end 42 and 43 is disposed in the bore 34 and is slidingly reciprocated by means of a linear actuator screw 44 driven by a step motor 46.

A sleeve bearing 47 having a sealing O-ring gasket 48 constituting a stationary annular sealing member is disposed at the first end 37 and an O-ring g gasket 49 constituting a displaceable annular sealing member is disposed at the slide rod end 42, the gaskets 48 and 49 sealingly the peripheral surfaces 36 and 41 to define a displacement volume 51 vented by a vent 52. The displacement volume 51 has a volume equal to a product of a cross sectional area between the surfaces 36 and 41 defined by π(a²-b²) and the distance between the gaskets 48 and 49.

The slide rod 39 is slidingly reciprocable between first and second positions respectively toward and away from the gasket 48 whereupon the displacement volume 51 has a minimum value when the gaskets 48 and 49 are adjacent in which case a major portion of the slide rod 39 is exterior to the bore 34 and a maximum value when the gaskets 48 and 49 are remote from one another. In operation, the gasket 49 moves to reduce the volume of the displacement volume 51 to issue an outgoing flow of displacement gas therefrom on a downstroke of the slide rod 39 from its second position to its first position and the gasket 49 moves to increase the volume of the displacement volume 51 to draw an incoming flow of displacement gas thereinto on an upstroke of the slide rod 39 from its first position to its second position.

The bore 34 and the slide rod 39 typically have diameters in the range of about 2-10 mm and which differ in the range of about 0.1-1 mm such that the cross section area is in the order of about 1-10 mm². The threading on actuator screw 44 is designed such that each step of the motor 45 causes an incremental movement of the slide rod 39 of about 0.0005-0.005 inches. The motor 45 is typically driven at a rate of about 20-300 steps per second. The pump has a displacement valve incremental changeable in the order of 0.01-0.5 μl.

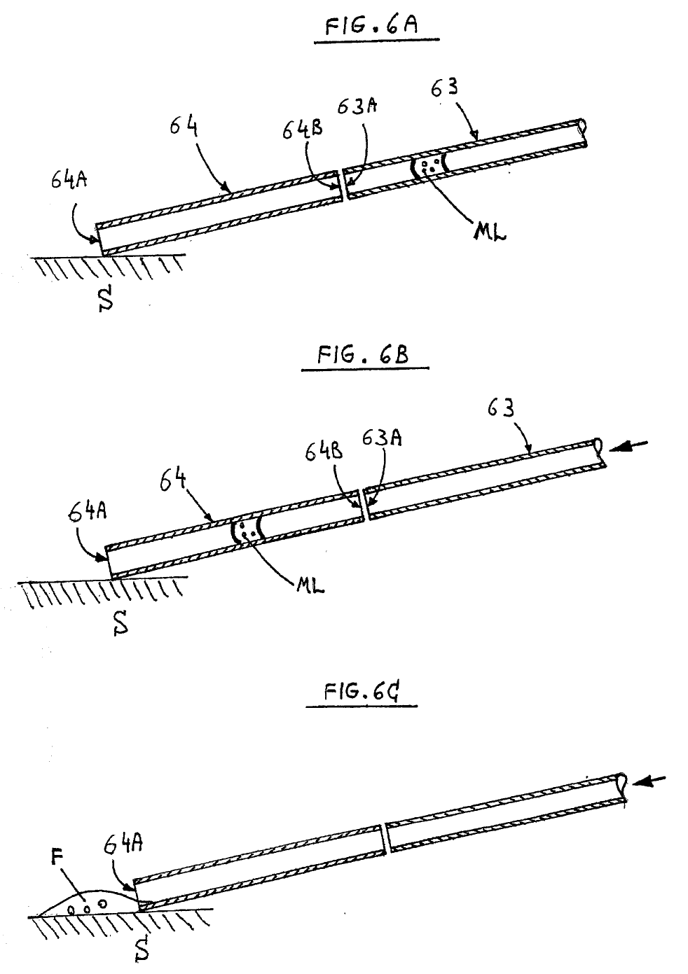

FIGS. 6A to 6C illustrate an extension catheter according to a further aspect of the present invention, which for example may by stationary mounted inside a human body and directed to a desired area of a tissue S where the delivery of a plurality of cells needs to be performed, to discharge there MLs with such cells, one-by-one, from a distal end of the extension catheter. The extension may be slightly displaced at the distal end close to the targeted tissue in order for the droplets to be placed adjacent to one another. A NE is aspired into the catheter 63, in accordance with the above-identified description and FIGS. 2A-2D. This procedure will be done regularly in-vitro. Once the droplet has been aspired into the catheter 63, the distal end of the catheter 63A is hermetically connected to the proximal end of the extension 64B by means of a sealed connector or an additional valve, one exempla of which will be described in more detail below. The extension 64 as can be seen in FIG. 6A thus becomes a natural continuation of the catheter 63 through which a M1 is moved outwardly to be discharged at the targeted tissue from the distal end 64B of the extension.

Extension 64 can be inserted into the human body through a standard drain used in surgical procedure while its distal end (FIG. 6B 64A) is permanently directed to the transplanted tissue. Gas is inserted into catheter 63 by means described above, and the ML aspired by the catheter 63 and comprising the cell(s) gradually moves into the extension 64 (FIG. 6B). The movement continues until the ML reaches the distal end 64A of the extension 64, where it is then discharged (FIG. 6C), as a flattened droplet F (as detailed in FIGS. 2L-2H). This action may occur several times whilst for each time the extension 64 is slightly moved along the tissue S (FIG. 6C) to an adjacent transplantation location. This movement can, be performed in accordance with the mechanism which will be described in more detail below. After repeating the procedure several times, a monolayer or a multilayer of cells may be formed in the transplantation location.

Once the flattened drops are placed on the transplanted tissue, they may either fuse among themselves, in accordance with physical forces, to form a large flattened drop or, may be transplanted as separate drops (FIG. 7).

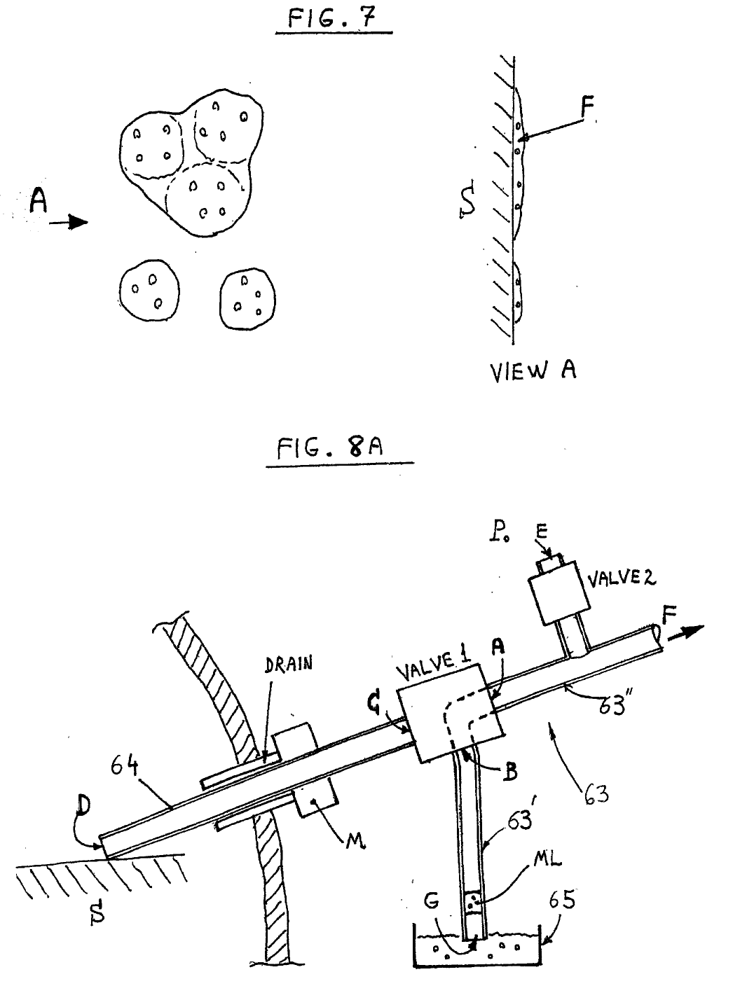

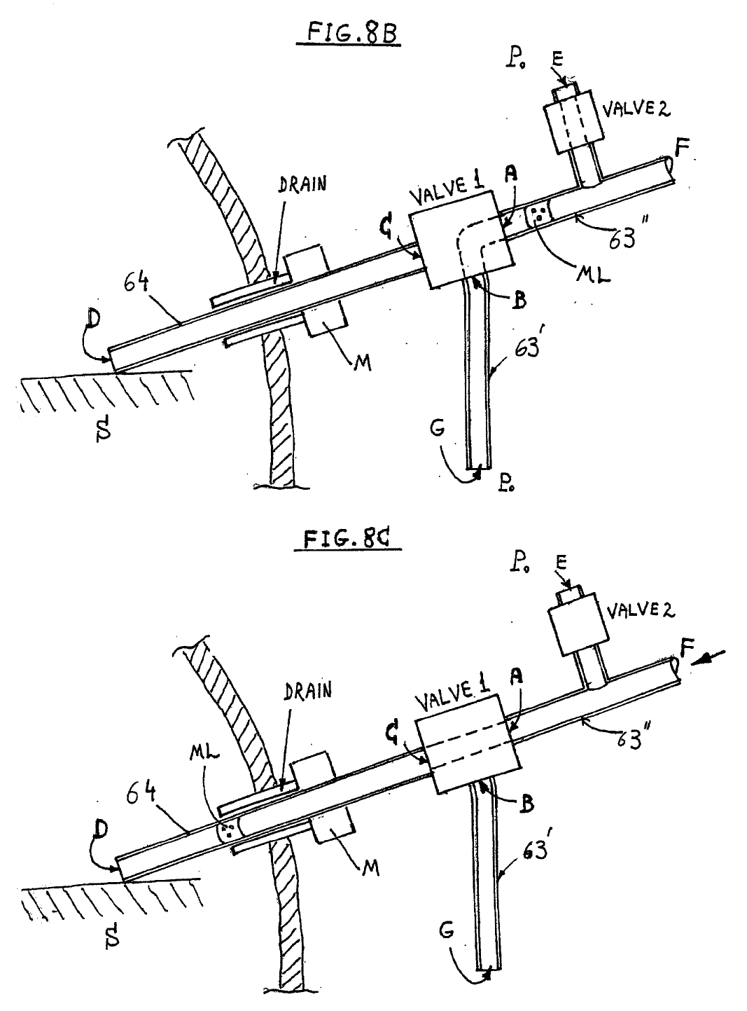

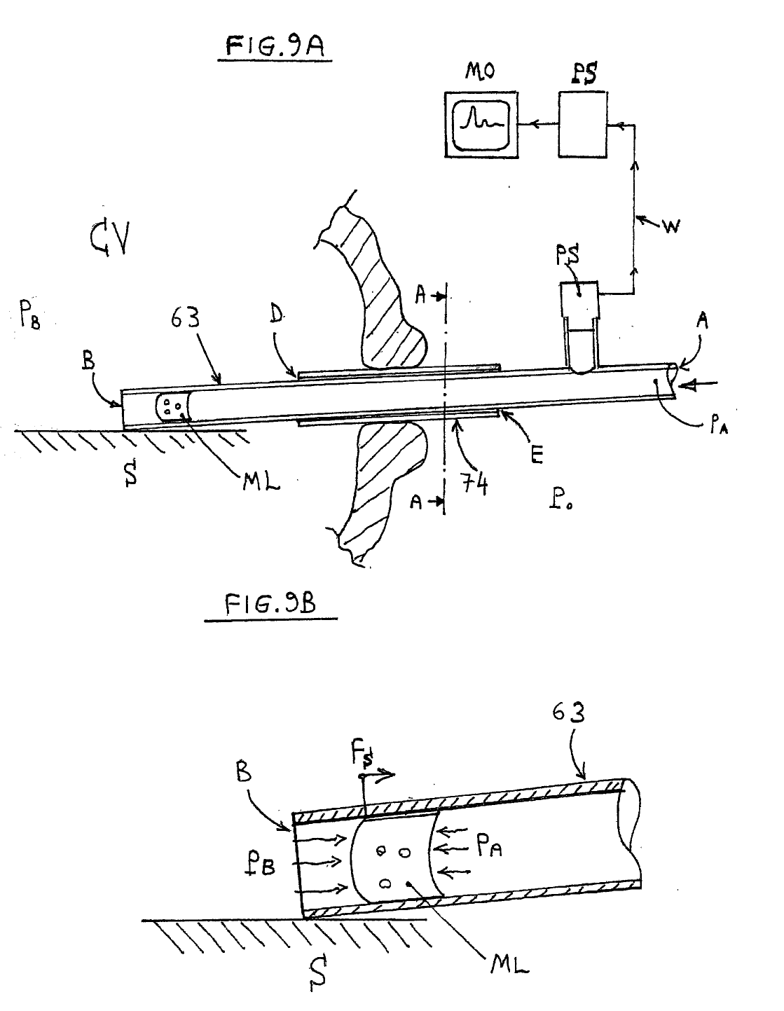

FIGS. 8A-8C illustrate a system of the present invention which comprises a connecting valve 1 and an arresting valve 2. Tile connecting valve 1 controls the movement of an ML to and from the catheter 63 and the extension 64, the catheter having an inlet section 63′ and an outlet section 63”. Valve 1 hermetically connects the catheter outlet section 63″ with tie catheter's inlet section 63′ and with the extension 64, and is adapted to switch between the two, to provide an aspiration path from the inlet end G of the catheter to its outlet section 63″, and a discharge path from the outlet section 63″ to the extension 64. The arresting valve 2 is connected to the outlet section 63′ of the catheter to provide there a full arrest of the IL at a predetermined location, to terminate the aspiration procedure and to start the discharge procedure of the ML.

FIG. 8A illustrates the aspiration of a ML from dish 65, which includes culture medium and cells, through the inlet end G of the catheter inlet section, 63′, Once the ML including the cultured cells is seen (through a microscope) to have entered the catheter inlet end. G, the dish is removed and the ML continues to move towards valve 1, and therethrough, towards and into the outlet section 63″ of the catheter, as shown in, FIG. 8B, by means of an outgoing flow of displacement gas creating a negative pressure at the proximal end F of the catheter. Once the NL passes the position A, and reaches a location between the valve I and valve 2, an arresting step is performed.

The arresting step as shown in FIG. 83 occurs when the valve 2 is switched to ON position, to be opened to the atmosphere (P₀ atmospheric pressure) at its outlet E, therefore canceling the pressure differential between the proximal end F and the inlet end G of the catheter sections 63′ and 63″. The ML is located within catheter outlet section 63″ and comes to a full arrest both said ends are exposed to the atmospheric pressure.

FIG. 8C illustrates a discharge procedure which starts when the valve 2 is switched back to OFF position and valve 1 disconnects catheter inlet section 63′ from the catheter outlet section 63″ and hermetically connects the latter to the extension 64. A slowly incoming flow of displacement gas creating a positive pressure is provided at the proximal end F of the catheter, which pushes the ML through valve 1 into extension 64. The NL keeps moving in the extension until it suddenly ruptures from the distal end D of the extension, whereby most of the ML containing cell(s) is discharged as a droplet on the targeted tissue S.

The system further comprises a control device M, which controls minor movements of the distal end of the extension 64 at the targeted tissue, to deliver thereto a plurality of MLs.

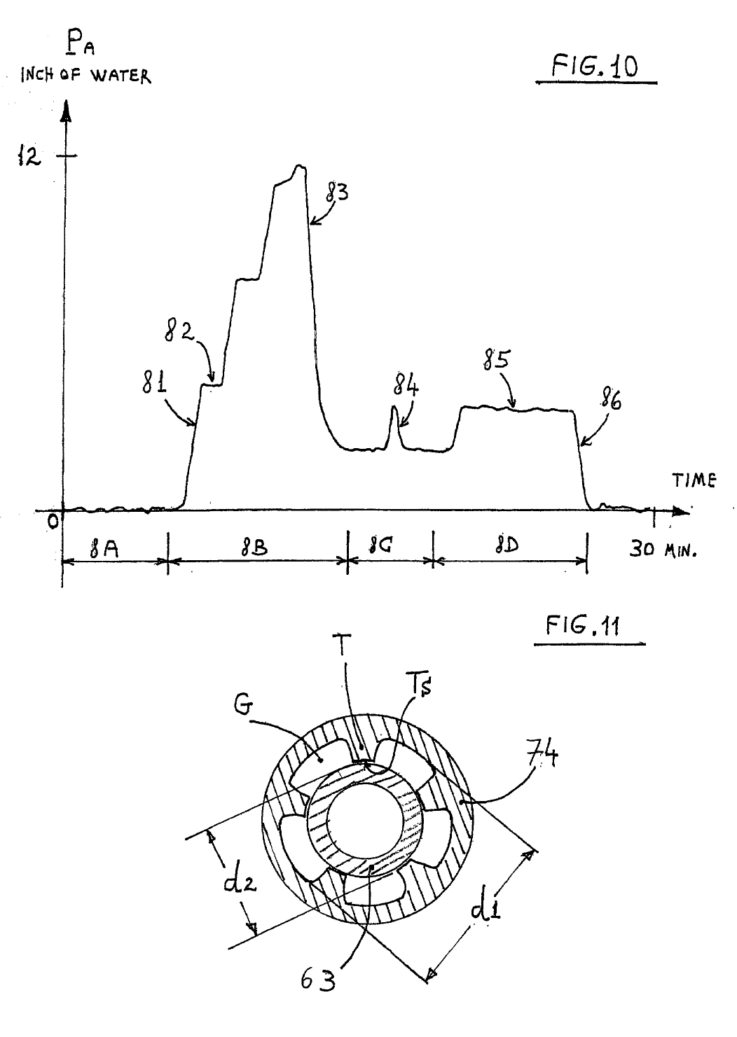

FIG. 9A illustrates how a catheter 63 according to the present invention may used for measuring pressure inside a Closed Volume (CV) of the human body, which may be filled with fluid, whereto a delivery of a NS is to be performed. The catheter 63 is connected to a Pressure Sensor (PS), is placed inside a guide 74 which is inserted into the CV of the human body e.g. cervix. The pressure sensor, for example a Piezoresistive Silicon Pressure Sensor, monitors the pressure inside the catheter 63 between the location of the ML and the proximal end A of the catheter. The sensor which is connected to a computer via cable W, translates the measured pressure to an Electric Potential. The screen MO shows the variations of the pressure during the time of the procedure.

When the catheter is inserted in the CV, its distal end will be filled with fluid that is present there. FIG. 9B illustrates the forces exerted on the NL during the process of die delivery to the targeted tissue S. PA is the only force which pushes the ML towards the tissue S. Oppositely directed forces are as follows:

F_{S}, includes a force created by the tension of the M, against the inner surface of the catheter, and a force of friction created as a result of shear stress between the ML and the inner surface of the catheter whenever the ML is moved;

P_{B} is a pressure created by the fluid in the CV;

With the catheter's inner cross-section area being designated as Ac, the equilibrium the forces acting o) the ML can be expressed by the following equation: P_{A}*A_{C}=P_{B}*A_{C+F}_{S}. Thus, though the pressure measurement is performed at the location of the catheter outside the body, the pressure at die catheter's end in, side the body is calculated.

FIG. 10 graphically illustrates pressure measured in a womb by monitoring as explained above throughout a typical ET procedure, which procedure should usually be performed when the muscle activities in a womb are the weakest and the pressure is the lowest measured. As seen in. FIG. 10, section 8SA of the graph corresponds to the atmospheric pressure measured before the procedure starts or after performing the arrest of the NL, as described above.

Section 8B illustrates the increasing pressure 81 due to the slow insertion of gas which pushes the ML towards the distal end of the catheter, in a manner described above, until the ML is discharged. When the is discharged and located on the targeted tissue, the pressure at the distal end of the catheter decreases rapidly at 83. If the pressure exceeds 12 Inch of Water without discharging the ML, then it is an indication that a problem such as bending the catheter inside the CV, blockade in the catheter with soft tissue or blood, had occurred. At any time of moving the MT towards the distal end of the catheter, the gas insertion may terminated and the MN will be arrested due to the pressure equilibrium designated as 82.

Section 8C of the graph demonstrates the cleaning of the catheter by blowing the displacement gas therethrough. In case, no fluid has been left in the catheter after the ML delivery procedure, the pressure will rapidly increase and decrease as shown at 84.

Section 8D illustrates a cleaning stage just like SC with the exception that the catheter contains tissue or blood etc., in which case a back flow of liquid may cause retained embryos hence the pressure is maintained at 85. Finally the catheter is removed from the CV and the pressure is dropped 86 back to the atmospheric pressure P₀. In general, sections B-D may last approximately 15 minutes.

FIG. 11 shows a cross section view of the catheter 63 inside the guide 74 of the present invention, along the line A-A in FIG. 9A. The guide 74 has a distal end D and a proximal end E, which is opened to the atmospheric pressure P₀. In addition, the guide is adapted to discharge the pressure from the CV. The guide is shaped in a Polar Pattern Groove with indentations G which form gaps between the indentations and the outer surface of the catheter 63. d1 is the inner diameter of the guide 74 defined by the indentations G and this diameter is in the range of 1-5 mm. d2 is the outer diameter of the catheter 63 and it is in the range of 0.5-4 mm. Fluid from the CV can flow through the gaps defined by the difference between the diameters d1 and d2, thereby reducing the pressure from the CV. The gap ranges between 0.5-3 mm. The guide 74 of the present invention is advantageous over a standard guide which does not have indentations, since the latter guide would compresses a catheter when entered therethrough into a CV, creating thereby a dynamic pressure exerted from the distal end of the catheter on the surface of the NL. This pressure may push the ML backwards whilst risking it to be discharged from the proximal end A of the catheter (FIG. 9A).

1. A method for delivery of cells substantially as described hereinbefore with reference to the accompanying drawings.

2. A catheter extension substantially as described hereinbefore with reference to the accompanying drawings.

3. A connection valve substantially as described hereinbefore with reference to the accompanying drawings.

4. An arresting valve substantially as described hereinbefore with reference to the accompanying drawings.

5. A monitoring system substantially as described hereinbefore with reference to the accompanying drawings.

6. A pressure discharge system substantially as described hereinbefore with reference to the accompanying drawings.

7. An apparatus for delivery of HES cells to a surface, comprising: a suction control unit; carrying catheter; a transfer control unit; and a portable casing for consecutive connection to the suction control unit and the transfer control unit by means of connectors; said casing including a pneumatic system connected to the catheter, said casing having a receptacle for accommodating the catheter, said pneumatic system comprising a control system for controlling the suction control unit, wherein each component is operably linked for delivery of HES cells to a surface.
